(19) United States
(12) Patent Application Publication
(10) Pub. No.: US 2009/0182036 A1
(43) Pub. Date: Jul. 16, 2009

(54) ANIMAL FOOD COMPOSITION COMPRISING GENISTEIN AND POLYUNSATURATED FATTY ACIDS

(76) Inventor: Stephanie J.M. Krammer-Lukas, Lörrach (DE)

Correspondence Address: NIXON & VANDERHYE, PC, 901 NORTH GLEBE ROAD, 11TH FLOOR, ARLINGTON, VA, 22203, US

(21) Appl. No.: 12/299,712

(22) PCT Filed: May 1, 2007
(86) PCT No.: PCT/EP07/03829

§ 371 Date: Nov. 5, 2008

(30) Foreign Application Priority Data
May 10, 2006 (EP) 06009635.1

(51) Int. Cl. A61K 31/352; A61P 25/00

(52) U.S. Cl. 514/456

(57) ABSTRACT

The invention relates to the use of nutraceutical compositions comprising as active ingredients genistein and at least one poly-unsaturated fatty acid for improving cognitive functions and/or for the treatment or prevention of age-related disorders in pets. The compositions are of primary interest for use in dog and cat food.

The present invention relates to a novel use of a nutraceutical composition for animals, especially for pets as dogs and cats, comprising as active ingredients genistein and polyunsaturated fatty acids.

More specifically, the invention relates to the use of such nutraceutical compositions as feed additives or nutraceuticals in order to improve cognitive functions and/or for the treatment or prevention of age-related disorders in animals, in particular in dogs and cats. There is an increasing interest in the development of compounds as well as nutraceuticals compositions that may be used to improve learning memory and alertness for example in elderly pets, and/or that may be used to treat mental disorders or to prevent the development of mental disorders.

In the context of this invention the term “disorder” also encompasses diseases.

The term nutraceutical as used herein denotes usefulness in both the nutritional and pharmaceutical field of application. Thus, the novel nutraceutical compositions can find use as supplement to feed and as pharmaceutical formulations for parenteral application which may be solid formulations such as capsules or tablets, or liquid formulations, such as solutions or suspensions. As will be evident from the foregoing, the term nutraceutical composition also comprises supplement compositions containing the aforesaid active ingredients (Inventive Ingredients) as well as feed and feedstuff including premixes used therefore, especially for pets, which contain the mixture of Inventive Ingredients.

The term “genistein” as used herein comprises the aglycone (4′, 5, 7-trihydroxyisoflavone) and derivatives thereof, e.g., genistein glycosides, genistein sulfates, genistein glucuronides. Genistein is a phytoestrogen belonging to the isoflavone class of flavonoid. It is abundant in soy bean and was reported to have antioxidant activities.

The term “polyunsaturated fatty acids” as used herein (herein also referred as PUFA) denotes a polyunsaturated fatty acid in an esterified (e.g., as triglycerides or ethyl esters) or a free form. Preferred poly-unsaturated fatty acids are those having from 16 to 24 carbon atoms, preferably from 18 to 22 carbon atoms, preferably with 18, 20 or 22 carbon atoms and having multiple unsaturated carbon-carbon double bonds.

Examples of such poly-unsaturated fatty acids are the known n-3 PUFA's. Preferred are for example poly-unsaturated acids such as n-3 PUFA, mainly as eicosapentaenoic acid (5,8,11,14,17-eicosapentaenoic acid, EPA) and docosahexaenoic acid (4,7,10,13,16,19-docosahexaenoic acid, DHA), or an omega-6-polyunsaturated fatty acid such as γ-linolenic acid (6,9,12-octadecatrienoic, GLA).

Cognition, broadly defined, refers to mental processes such as perception, awareness, learning, memory, and decision making. Cognition allows an animal to take in information about the environment, process, retain, and make decisions how to act. These mental processes cannot be measured directly. Different cognitive tasks have been developed to evaluate learning and memory in dogs. In order to better understand the behavioural consequences associated with cognitive changes in ageing, a series of behavioural reactivity tests to evaluate stimulus-evoked behaviour were developed. Based on the animal's response to the various stimuli, these tests can distinguish cognitively impaired aged dogs from those who successfully age.

In the following some tasks are summarized:
The human interaction tests:
   The test assesses the reaction of a dog to the presence of a person. A person familiar to the dog sits in the middle of a room while the dog is free to explore. Young dogs spend significantly more time in physical contact with the person than normal aged dogs. Cognitively unimpaired aged dogs spent a lot of time close to the person without actually making physical contact, while impaired aged dogs pay little attention to the person.
The silhouette and model dog test:
   These tests measure social responsiveness to other dogs. The silhouette tests uses a cardboard figure of a dog taped to the wall and the model dog tests uses a life-size plastic replica of a dog sitting in the centre of a room to assess social responsiveness.

Young dogs are more responsive to the artificial conspecifics showing significantly more investigative sniffing than both groups of old dogs.
The curiosity test:
   The test measures exploratory behaviour by allowing dogs to examine and play with a variety of toys. Young dogs explore and contact the novel objects significantly more than old dogs. The cognitively impaired aged dogs show almost no interest in toys.
The mirror test:
   It examines an individual animal's reaction to its mirror image. Young dogs and normal aged dogs have mild reactions to the reflection but habituated to the image fairly quickly. The cognitively impaired dogs spent significantly more time reacting to the reflection. Their response can include jumping and barking at the dog in the mirror.
Landmark discrimination learning tasks:
   The dogs are presented with two identical objects. To obtain food reward, the animals are required to respond selectively to the object closest to a specific external cue.
Oddity discrimination learning tasks:
   In these task the animal is presented with three objects, two identical and one different. To obtain a reward, the animal is required to respond to the odd object. Difficulty can be increased based on similarity of positive and negative objects.
Size discrimination learning task and reversal learning tasks:
   The test evaluates the animal's ability to learn to distinguish two objects that differ only in size in order to locate a food reward.

Aging involves a progressive deterioration and loss of the cellular processes and physiological functions of an organism that ultimately increase the likelihood of death. The aging process involves a number of molecular pathways such as oxidative stress, cellular stress resistance, neuroendocrine systems, nutrient sensing systems and insulin signaling.

Age related diseases and disorders in general can be grouped as follows:
Central nervous system disorders: The aging process often causes atrophic changes in the brain. There are substantial age-related declines in brain function, i.e., decrease in norepinephrine and dopamine synthesis.
Autonomic nervous system disorders: Since the homeostatic mechanisms slow and weaken during advancing age, changes are reflected in the alterations of sympathetic and parasympathetic responsiveness, i.e., decreased sensitivity of baroreceptor and change in thermoregulation.
Eye and ear disorders: Eye Disorders—Physiological changes of presbyopia and lens opacification subsequently cause decreased accommodation and increased susceptibility to glare. These physiological changes often result in decreased visual acuity as well as blindness. Ear Disorders—For the ear, the physiological change is decreased high frequency acuity, making it difficult to discriminate words if noise is present in the background. Consequently, there is deafness and a decrease in acoustic acuity.
Cardiovascular system disorders (diseases include hypertension, coronary artery disease.
Congestive heart failure as well as heart block or arrhythmia).
Respiratory system disorders (Respiratory diseases include emphysema, dyspnea, and hypoxia).
Gastrointestinal system disorders.
Endocrine system disorders (include the development of diabetes mellitus, thyroid dysfunction)
Hematological and immune system disorders.
Muscular and skeletal system disorders (osteoporosis)
Cancer.

And specific for pets the following diseases and disorders are known:
Oxidative damage is, besides plaque and neurofibrillary tangles deposition, another age-dependent type of pathology. Over the course of ageing and normal cellular metabolism, oxidants are produced, that if not reduced by endogenous antioxidants, can damage proteins, lipids and nucleotides. These forms of damage can be measured by biochemical assays for lipid peroxidation (malondialdehyde), protein carbonyl formation, enzyme dysfunction and the accumulation of DNA/RNA oxidative damage. In canine, these markers of oxidative damage progressively increase with age. Further preliminary evidence suggests that oxidative damage to RNA may precede the accumulation of β-amyloid.
β-amyloid deposit: One form of neuropathology that was described in dogs over 40 years ago is the development of senile plaques. Plaques are deposits of a toxic peptide called β-amyloid (Aβ) in the space between neurons. Aβ is derived from a longer β-amyloid precursor protein through the activity of two enzymes: β-secretase/BACE or γ-secretase. These enzymes can produce either Aβ that is 40 or 42 amino acids long. The longer species Aβ 1-42 aggregates more rapidly then the shorter Aβ 1-40. The earliest senile plaques contain predominantly Aβ1-42. Aβ that is associated with blood walls, called Aβ angiopathy, almost entirely consist of the shorter 40 amino acid long peptide. Aβ is deposited in the dog brain with age in a specific pattern: the prefrontal cortex appears to develop Aβ earlier than other regions with dogs over the age of 10 years. This may suggest that prefrontal cortex functions, such as reversal learning, behavioural rigidity and preservative behaviours may be early manifestations of Aβ pathology. As in human brain ageing, the extent of senile plaque formation is higher in dogs with cognitive dysfunction. The extent and location of Aβ-pathology is associated with performance on specific learning tasks, which are thought to be mediated by these same brain regions (Head, 2002).
Neurofibrillary tangles. They are formed by the intracellular accumulation of tau protein, which makes up the cytoskeleton of neurons. In Alzheimer's disease, tau protein becomes hyperphosphorylated and forms paired helical filaments, which fill the cytoplasm and lead to neuron dysfunction (Cotman, 2002).

The statement above can be summarized as follows:
Due to the advances in veterinary medical care and changes in socioeconomic status, dogs in domestic settings are living longer today then in past times.
An estimated 18 million pet dogs in the United States alone are more than 7 years of age.
Increased life span appears to be inherently associated with age-related disease process such as (cancer, renal disease, and) cognitive decline.
As the cognitive aging process is associated with progressive decline in cellular function, it follows that a larger number of older dogs may also be at risk to develop behavioral changes related to cognitive decline.
These behavioral alterations are often manifested as disorientation (D), altered interactions with the family members (I), disruptions in sleep (S), loss of house training (H), and altered activity levels (A). Collectively, these behavioral attributes may be identified by the acronym “DISHA”.

Surprisingly, it has now been found that the present compositions act on different critical signaling pathways involved in aging and hence delay aging and age-related diseases more potently than the individual components.

More precisely it has been found that compositions containing as active ingredients genistein and PUFA's may be useful to improve cognitive function such as perception, awareness, learning, memory, and decision making.

Moreover it has now been found that compositions containing genistein in combination with PUFA's, have a significant additive and synergistic effect as nutraceutical in preventing and treating of central nervous system disorders, autonomic nervous system disorders, eye and ear disorders, cardiovascular system disorders, congestive heart failure, gastrointestinal system disorders, hematological and immune system disorders, muscular and skeletal system disorders and cancer in pets.

In yet another aspect, the present invention relates to the use of a combination of genistein and at least one poly-unsaturated fatty acid for the manufacture of a composition as feed additives or nutraceuticals to improve cognitive functions and/or for the treatment or prevention of age-related disorders in animals, in particular in dogs and cats.

The present invention further refers to a method to improve cognitive functions and/or for the treatment or prevention of age-related disorders in animals, in particular in dogs and cats, which comprises administering to a pet an effective amount of said composition.

Advantageous embodiments of the invention become evident from the dependent claims.

Genistein and PUFA may be incorporated into conventional pet food e.g., into dry pet food by spraying a solution, for example an aqueous solution containing the Inventive Ingredients on the food composition while thoroughly mixing the composition, or by adding the Inventive Ingredients to the dough. Inventive Ingredients may be added simultaneously, e.g. at the same time and even as a premix, or consecutively as single Inventive Ingredient at a time or as a premix. Premixes may also include one or more of the other components of the final composition.

The nutraceutical compositions of the present invention may be in the form of a premix and may contain Genistein in an amount sufficient to administer to an adult dog (weighing about 20 kg) a dosage from about 0.1 mg/day to about 800 mg/day, preferably from about 0.5 mg/day to about 100 mg/day and PUFA's in an amount sufficient to administer to an adult dog (weighing about 20 kg) a dosage from about 1 mg/day to about 5000 mg/day, preferably from about 3 mg/day to about 2500 mg/day.

In the context of this invention “treatment” also encompasses co-treatment as well as prevention. “Prevention” can be the prevention of the first occurrence (primary prevention) or the prevention of a reoccurence (secondary prevention).

The following Examples illustrate the invention further. The Examples are for illustrative purposes only and are not intended, nor should they be construed, as limiting the invention in any manner. Those skilled in the art will appreciate that variations and modifications can be made without violating the spirit or scope of the invention.

Said composition may be provided in the form of a concentrate, for example as a simple powdery mixture of its components; or in the form of granules as are obtained for example by spray drying an aqueous slurry of the components or by extruding the mixture; or in the form of tablets as are obtained by compressing the powder into tablets with conventional tableting methods and machinery.

The pet food according to the present invention may be based on any conventional pet food. There is a wide range of pet foods available which may be grouped into (a) complete diets, (b) complementary diets, and (c) snacks and treats. Complete diets may be fed in addition to water for an extended period as the sole source of nutrients and will provide for all the energetic and nutrient needs of the animal and the physiological state for which it is intended. Complementary diets normally are not sufficient to ensure that all nutrient and energy requirements are met unless fed in combination with another foodstuff or diet. Snacks and treats are appetizers or for occasional feeding and are considered as complementary products. There are, however, a number of products available intended to form part of the daily diet or playing a role in animal well-being.

The pet food of the present invention may be in a dry, canned, semi-moist or baked form. Typical components of such compositions, in addition to Inventive Ingredients, are crude protein, crude fat, carbohydrates (NfE), starch, crude fibers, and ash, further on minerals, trace elements, vitamins, fatty acids, protein and amino acids, choline, camitin, dietary fiber and substances required for balanced diets of the different animal species. Basic ingredients of such food compositions are
Crude Protein including proteins and N-containing compounds of non-proteinaceous nature, e.g. acid amides, amines, free amino acids, ammonium salts, alkaloids;
Crude Fat including neutral fats, lipoids (phospho-, sphingolipids, steroids) and other ethersoluble compounds;
N-free Extractions (NFE) including polysaccharides (starch, glycogen), soluble saccharides (glucose, fructose, saccharose, lactose, maltose and oligosaccharides), and soluble fractions of cellulose, hemicellulose, lignin and pectines;
Crude Fibers including insoluble fractions of cellulose, hemicellulose, lignin and other components of the cell wall like suberin, cutin etc.;
Ash including minerals (macrominerals such as calcium, phosphorus, sodium, chloride, potassium, magnesium, and microminerals, i.e., trace elements, such as iron, copper manganese, zinc, iodine, selenium,) and further inorganic substances e.g. silicate.
Vitamins including vitamins A, B1, B2, B6, B12, D, pantothenic acid, niacin, folic acid, linolic acid and choline.

Further components may, e.g. L-carnitine, chondroitin sulfate, glucosamine, glutamine/glutamic acid, arginine, taurine and hydroxyproline.

Typical components which provide the ingredients for a dog food composition, in addition to Inventive Ingredients, comprise, e.g., chicken/beef/turkey, liver, broken pearl barley, ground corn, brute fat, whole dried egg, fowl protein hydrolyzate, vegetable oil, calcium carbonate, choline chloride, potassium chloride, iodinized salt, iron oxide, zinc oxide, copper sulfate, manganese oxide, sodium selenite, calcium iodate, provitamin D, vitamin B 1, niacin, calcium panthothenate, pyridoxin hydrochloride, riboflavin, folic acid, vitamin B12.

Typical components which provide the ingredients for a cat food composition, in addition to Inventive Ingredients, comprise beef, chicken meat, dried chicken liver, lamb meat, lamb liver, pork, turkey meat, turkey liver, poultry meal, fish meal, fowl protein hydrolysate, animal fats, plant oils, soy bean meal, pea bran, maize gluten, whole dry egg, ground corn, corn flour, rice, rice flour, dry sugar beet molasses, fructooligosaccharides, soluble fibers, plant gums, cellulose powder, clay, bakers yeast, iodized sodium chloride, calcium sulfate, sodium triphosphate, dicalcium phosphate, calcium carbonate, potassium chloride, choline chloride, magnesium oxide, zinc oxide, iron oxide, copper sulfate, iron sulfate, manganese oxide, calcium jodate, sodium selenite, provitamin D, thiamine, niacin, calcium pantothenate, pyridoxine hydrochloride, riboflavin, folic acid, vitamin B 12, taurin, L-camitine, caseine, D-methionine.

Wet pet food contains between about 70 and about 85% moisture and about 15 and about 25% dry matter.

A typical wet food for adult dogs may, e.g. comprise, in addition to Inventive Ingredients, at minimum 24% protein, 15% fat, 52% starch, 0.8% fiber, 3% linolic acid, 0.6% calcium, 0.5% phosphorus, the Ca:P ratio being 1:1, 0.2% potassium, 0.6% sodium, 0.09% chloride, 0.09% magnesium, 170 mg/kg of iron, 15 mg/kg of copper, 70 mg/kg of manganese, 220 mg/kg of zinc, 4 mg/kg of iodine, 0.43 mg/kg of selenium, 74000 IU/kg of vitamin A, 1200 IU/kg of vitamin D, 11 mg/kg of vitamin B 1, 6 mg/kg of riboflavin, 30 mg/kg of pantothenic acid, 20 mg/kg of niacin, 4.3 mg/kg of pyridoxine, 0.9 mg/kg of folic acid, 0.2 μg/kg of vitamin B 12, 2500 mg/kg of choline, 2500 mg/kg cholin, all percentages being based on dry weight of the total food composition.

A typical wet food for adult cats may, e.g. comprise, in addition to Inventive Ingredients, at minimum 44% protein, 25% fat, 20% starch, 2.5% fiber, 0.8% calcium, 0.6% phosphorus, 0.8% potassium, 0.3% sodium, 0.09% chloride, 0.08% magnesium, 0.25% taurin, 170 mg/kg of iron, 15 mg/kg of copper, 70 mg/kg of manganese, 220 mg/kg of zinc, 4 mg/kg of iodine, 0.43 mg/kg of selenium, 74000 IU/kg of vitamin A, 1200 IU/kg of vitamin D, 11 mg/kg of vitamin B 1, 6 mg/kg of riboflavin, 30 mg/kg of pantothenic acid, 20 mg/kg of niacin, 4.3 mg/kg of pyridoxine, 0.9 mg/kg of folic acid, 0.2 μg/kg of vitamin B12, 2500 mg/kg of choline, 2500 mg/kg cholin, all percentages being based on dry weight of the total food composition.

Dry pet food contains between about 6 and about 14% moisture and about 86% or more dry matter.

A typical dry food for adult dogs may, e.g. comprise, in addition to Inventive Ingredients, at minimum 25% protein, 12% fat, 41.5% starch, 2.5% fiber, 1% linolic acid, 1% calcium, 0.8% phosphorus, the Ca:P ratio being 1:1, 0.6% potassium, 0.35% sodium, 0.09% chloride, 0.1% magnesium, 170 mg/kg of iron,35 mg/kg of copper, 70 mg/kg of manganese, 220 mg/kg of zinc, 4 mg/kg of iodine, 0.43 mg/kg of selenium, 15000 IU/kg of vitamin A, 1200 IU/kg of vitamin D, 11 mg/kg of vitamin B1, 6 mg/kg of riboflavin, 30 mg/kg of pantothenic acid, 20 mg/kg of niacin, 4.3 mg/kg of pyridoxine, 0.9 mg/kg of folic acid, 0.2 μg/kg of vitamin B12, 2500 mg/kg of choline, all percentages being based on dry weight of the total food composition.

A typical food for adult cats may, e.g. comprise, in addition to Inventive Ingredients, at minimum 32% protein, 15% fat, 27.5% starch, 11% dietetic fibers, 4.5% fiber, 3.4% linolic acid, 0.08% arachionic acid, 0.15% taurin, 50 mg/kg L-camitin, omega 6/3 =5, 1 % calcium, 0.8% phosphorus, the Ca:P ratio being at least 1:1, 0.6% potassium, 0.4% sodium, 0.6% chloride, 0.08% magnesium, 190 mg/kg of iron, 30 mg/kg of copper, 60 mg/kg of manganese, 205 mg/kg of zinc, 2.5 mg/kg of iodine, 0.2 mg/kg of selenium, 25000 IU/kg of vitamin A, 1500 IU/kg of vitamin D, 20 mg/kg of vitamin B1, 40 mg/kg of riboflavin, 56 mg/kg of pantothenic acid, 153 mg/kg of niacin, 14 mg/kg of pyridoxine, 3.2 mg/kg of folic acid, 0.2 mg/kg of vitamin B 12, 3000 mg/kg of choline, all percentages being based on dry weight of the total food composition.

Dry food may be prepared, e.g., by screw extrusion including cooking, shaping and cutting of raw ingredients into a specific kibble shape and size in a very short period of time, while simultaneously destroying detrimental micro-organisms. The ingredients may be mixed into homogenous expandable dough and cooked in an extruder (steam/pressure) and forced through a plate under pressure and high heat. After cooking, the kibbles are then allowed to cool, before optionally being sprayed with a coating which may include liquid fat or digest including liquid or powdered hydrolyzed forms of an animal tissue such as liver or intestine from, e.g., chicken or rabbit. Hot air drying then reduces the total moisture content to 10% or less.

Canned (wet) food may be prepared, e.g., by blending the raw ingredients including meats and vegetables, gelling agents, gravies, vitamins, minerals and water. The mix is then fed into cans on a production line, the lids are sealed on and the filled cans are sterilized at a temperature of about 130° C. for about 50 to 100 min.

A typical formulation for a dog feed composition is shown in the following table.

| | |
|---|---|
| Genistein | between 1 and 300 mg/kg diet, preferably up to 100 |
| | mg/kg diet, preferably up to 75 mg/kg diet (Bonistein ™) |
| DHA | high 5.5 to moderate 1.9 to low 0.2 g/kg dry matter |
| | (ROPUFA ® ‘30’ n-3 INF Oil) |
| EPA | high 5.0 to moderate 1.9 to low 0.2 g/kg dry matter |
| | (ROPUFA ® ‘30’ n-3 INF Oil) |
| ARA | high 0.5 to moderate 0.3 to low 0.2 g/kg dry matter |
| | (ROPUFA ® ‘12’ n-6 ARA Powder) |
| Vitamin E | 500 mg/kg diet |
| Vitamin C | 300 mg/kg diet |
| Beta-carotene | 50 mg/kg |
| Vitamin B₁ | 20 mg/kg |
| Vitamin B₆ | 14 mg/kg |
| Vitamin B₁₂ | 0.05 mg/kg) |

The following Examples illustrate the invention further.

### EXAMPLE 1

Commercial dry dog food (Hill's Science diet “Canine Maintenance dry” for dogs as supplied by Hill's Pet Nutrition GmbH, Liebigstrasse 2-20, D-22113) is sprayed/drugged with genistein and ROPUFA® (as supplied by DSM Nutritional Products) in an amount sufficient to administer to a subject a daily dose of 0.1 mg to 3 mg genistein and 4 mg to 120 mg ROPUFA per kg body weight. Further Vitamin C and E and β-carotene are incorporated in an amount sufficient to provide 30 mg vitamin C/kg, and 300 IU vitamin E/kg and 280 mg β-carotene/kg in the final food composition before extruding the entire blend. The food composition is dried to contain dry matter of about 90% by weight.

### EXAMPLE 2

Commercial wet dog food (Hill's Science diet “Canine Maintenance wet” for dogs as supplied by Hill's Pet Nutrition GmbH, Liebigstrasse 2-20, 22113 Hamburg, Germany) is sprayed/drugged with genistein and ROPUFA® (as supplied by DSM Nutritional Products) in an amount sufficient to administer to a subject a daily dose of 0.1 mg to 3 mg genistein and 4 mg to 120 mg ROPUFA per kg body weight. Further Vitamin C and E and β-carotene are incorporated in an amount sufficient to provide 30 mg vitamin C/kg, and 300 IU vitamin E/kg and 280 mg β-carotene/kg in the final food composition before cooking the entire blend. The food composition is dried to contain a dry matter of about 90% by weight.

### EXAMPLE 3

Commercial dog treats (Mera Dog “Biscuit” for dogs as supplied by Mera Tiernahrung GmbH, Marienstrasse 80-84, 47625 Kevelaer-Wetten, Germany) are sprayed/drugged with genistein and ROPUFA® (as supplied by DSM Nutritional Products) in an amount sufficient to administer to a subject a daily dose of 0.1 mg to 3 mg genistein and 4 mg to 120 mg ROPUFA per kg body weight. Further Vitamin C and E and β-carotene are incorporated in an amount sufficient to provide 30 mg vitamin C/kg, and 300 IU vitamin E/kg and 280 mg β-carotene/kg in the final food composition before extruding the entire blend. The food composition is dried to contain a dry matter of about 90% by weight.

### EXAMPLE 4

Commercial dry cat food (Hill's Science diet “Feline Maintenance dry” for cats as supplied by Hill's Pet Nutrition GmbH, Liebigstrasse 2-20, D-22113) is sprayed/drugged with genistein and ROPUFA® (as supplied by DSM Nutritional Products) in an amount sufficient to administer to a subject a daily dose of 0.1 mg to 3 mg genistein and 4 mg to 120 mg ROPUFA per kg body weight. Further Vitamin C and E and β-carotene are incorporated in an amount sufficient to provide 30 mg vitamin C/kg, and 300 IU vitamin E/kg and 280 mg β-carotene/kg in the final food composition before extruding the entire blend. The food composition is dried to contain a dry matter of about 90% by weight.

### EXAMPLE 5

Commercial wet cat food (Hill's Science diet “Feline Maintenance wet” for cats as supplied by Hill's Pet Nutrition GmbH, Liebigstrasse 2-20, D-22113) is sprayed/drugged with genistein and ROPUFA® (as supplied by DSM Nutritional Products) in an amount sufficient to administer to a subject a daily dose of 0.1 mg to 3 mg genistein and 4 mg to 120 mg ROPUFA per kg body weight. Further Vitamin C and E and β-carotene are incorporated in an amount sufficient to provide 30 mg vitamin C/kg, and 300 IU vitamin E/kg and 280 mg β-carotene/kg in the final food composition before cooking the entire blend. The food composition is dried to contain a dry matter of about 90% by weight.

### EXAMPLE 6

Commercial cat treats (Whiskas Dentabits for cats as supplied by Whiskas, Masterfoods GmbH, Eitzer Str. 215, 27283 Verden/Aller, Germany) are sprayed/drugged with genistein and ROPUFA® (as supplied by DSM Nutritional Products) in an amount sufficient to administer to a subject a daily dose of 0.1 mg to 3 mg genistein and 4 mg to 120 mg ROPUFA per kg body weight. Further Vitamin C and E and β-carotene are incorporated in an amount sufficient to provide 30 mg vitamin C/kg, and 300 IU vitamin E/kg and 280 mg β-carotene/kg in the final food composition before extruding the entire blend. The food composition is dried to contain a dry matter of about 90% by weight.

1. A nutraceutical composition, which is a pet food or a supplement composition for a pet food, particularly for dogs or cats, characterized in that said composition comprises
(a) genistein and
(b) at least one poly-unsaturated fatty acid.

2. A composition according to claim 1, characterized in that component (b) is a poly-unsaturated fatty acid with 16 to 24 carbon atoms, preferably with 18 to 22 carbon atoms, preferably with 18, 20 or 22 carbon atoms, containing multiple unsaturated carbon-carbon double bonds.

3. A composition according to claim 2, characterized in that component (b) is an n-3 poly-unsaturated fatty acid.

4. A composition according to claim 1, characterized in that component (b) is selected from eicosapentaenoic acid, docosapentaenoic acid and docosahexaenoic acid.

5. A composition according to claim 1, characterized in that component (b) is selected from (cis-)5,8,11,14,17-eicosapentanoic acid and (cis-) 4,7,10,13,16,19-docosahexaenic acid.

6. The use of genistein and at least one poly-unsaturated fatty acid for the manufacture of a composition for use as feed additives or nutraceuticals to improve cognitive functions and/or for the treatment or prevention of age-related disorders in animals, in particular in dogs and cats.

7. Use according to claim 6, in order to improve learning memory and alertness.

8. The use as in claim 6, wherein said genistein being used in an amount sufficient to provide a daily dosage of 0.1 mg to about 3 mg per kg body weight and the at least one poly-unsaturated fatty acid being used in an amount sufficient to provide a daily dosage of 4 mg to about 120 mg per kg body weight.

9. The use as in claim 6, in the manufacture of a dog feed.

10. A method for improving cognitive functions and/or for the treatment or prevention of age-related disorders in animals, in particular in dogs and cats, which comprises administering to a pet an effective amount of a composition according to claim 1.

11. A method as in claim 10 wherein the pet is a dog or cat.

12. A method as in claim 10 wherein from about 0.1 mg to about 3 mg of genistein per kg body weight and day and wherein from about 4 mg to about 120 mg of the at least one poly-unsaturated fatty acid per kg body weight and day is administered.
